(19) United States
(12) Patent Application Publication
(10) Pub. No.: US 2024/0216659 A1
(43) Pub. Date: Jul. 4, 2024

(54) MICROSTRUCTURE CARTRIDGE AND MICROSTRUCTURE APPLICATOR

(71) Applicant: SNVIA CO., LTD., Busan (KR)

(72) Inventors: Kang Oh LEE, Busan (KR); Seung Soo LEE, Busan (KR)

(73) Assignee: SNVIA CO., LTD., Busan (KR)

(21) Appl. No.: 18/570,924

(22) PCT Filed: Jun. 15, 2022
(86) PCT No.: PCT/KR2022/008452

§ 371 Date: Dec. 15, 2023

(30) Foreign Application Priority Data
Jun. 17, 2021 (KR) 10-2021-0078456

(51) Int. Cl. A61M 37/00

(52) U.S. Cl. CPC A61M 37/0015; A61M 2037/0023; A61M 2205/273; A61M 2209/088

(57) ABSTRACT

A microstructure cartridge is disclosed. The microstructure cartridge comprises: a base plate including a first plate region that is inserted into a microstructure applicator, a second plate region that is not inserted into the microstructure applicator adjacent to the first plate region, and a bending or cutting guide line forming a boundary between the first plate region and the second plate region; and microneedles located in the first plate region, wherein the second plate region is configured to be bent or separated by the guide line as a boundary.

### FIELD

The present disclosure relates to a microstructure cartridge and a microstructure applicator, and more particularly, to a microstructure cartridge and a microstructure applicator that may easily identify whether a microneedle has been used.

### DESCRIPTION OF RELATED ART

Since the skin covers the outside of the living body, the skin is composed of three layers of epidermis, dermis, and subcutaneous fat layers from the outside to a human organ protecting muscles and organs in the body, and has various functions such as body temperature adjustment and function as a barrier to an external environment.

The epidermis occupies most of the keratinocytes, in addition to melanocytes, Langerhans cells, indeterminate cells, Merkel cells. The stratum corneum is a first protective film of a human body that performs a barrier function (a selective permeation barrier, a physical barrier film, an ultraviolet protective film, a harmful substance defense film, a microbial barrier film), a moisturizing function, an immune function, an antioxidant function, an anti-inflammatory function, an antibacterial function, a regeneration function, and the like.

Most active substances do not pass through the waterproof film in the presence of a waterproof film on the lower part of the stratum corneum and the upper part of the granular layer. To overcome this situation, 1) the particle size of the active material is reduced, or the nano-carrier is added or passed, 2) the waterproof film is passed through a physical, chemical, mechanical method, or 3) the formulation is configured in the form of a liposome, a nanoemulsion, and a nanoparticle to allow the stratum corneum to pass through the stratum corneum. Products that maintain an intercellular lipid layer which is the most important to the barrier function and moisturizing function of the stratum corneum and penetrate the active material must be amphiphilic (nanooil water form), and may pass through a lipid layer between cells having a size of 100 nm or less and a molecular weight of 500 Daltons.

As such, the delivery of the drug into the body through the skin is very difficult to deliver the desired substance if it does not pass through the stratum corneum, which is a primary barrier. Ointment and cream formulations developed to treat dermatological diseases are designed to allow drug delivery through these stratum corneum and to exhibit appropriate effects on target sites. However, as described above, even if the molecular weight of the drug is more than 500 Daltons or the structure thereof is difficult to pass through the stratum corneum, the materials which are difficult to pass through the stratum corneum are delivered to the inside of the skin, and thus it is not easy to expect the desired purpose and efficacy thereof.

Therefore, in order to more efficiently deliver a desired drug by forming an artificial channel in the stratum corneum, a microneedle technology is commercialized and used.

Generally, in a configuration of a microneedle patch, a plurality of microneedles having a fine needle shape are fixed to a base, the base is fixed to an attachment band, and the attachment band is attached to the skin, wherein the microneedle acts as a passage through which the active ingredient is delivered into the skin.

However, in the conventional microneedle patch, the drug is delivered through the microneedle. Thus, it is difficult to check whether the microneedle has been used after the drug delivery, and thus, the microneedle that has already been used is reused. This is problematic.

### DISCLOSURE

### Technical Purpose

Accordingly, a purpose to be achieved by the present disclosure is to provide a microstructure cartridge and a microstructure applicator which may easily check whether a microneedle has been used, thereby preventing a situation in which the microneedle that has already been used is reused, without confusion about whether the microneedle has been used.

### Technical Solution

A microstructure cartridge according to one embodiment of the present disclosure comprises: a base plate including: a first plate region to be inserted into a microstructure applicator; a second plate region adjacent to the first plate region and constructed not to be inserted into the microstructure applicator; and a bending or cutting guide line as a boundary between the first plate region and the second plate region; and a microneedle disposed on the first plate region of the base plate, wherein the second plate region is constructed to be bent or cut along the guide line.

In one embodiment, the guide line is formed in a groove extending linearly along one direction.

In one embodiment, the guide line includes a linear arrangement of holes arranged so as to be spaced from each other by a predetermined spacing along one direction.

A microstructure applicator according to one embodiment of the present disclosure comprises: a housing having a sidewall having a predetermined height, wherein upper and lower ends of the housing are opened; and a cartridge receiving portion having a cartridge receiving surface for receiving the microstructure cartridge as described above therein, wherein the cartridge receiving surface is constructed to be inserted into the housing, wherein the second plate region is exposed to an outside while not being received in the cartridge receiving portion, wherein when the cartridge receiving portion is received in the housing, the second plate region is engaged with the sidewall so as to be bent or cut.

In one embodiment, one side of the cartridge receiving surface is hingedly connected to the sidewall such that the cartridge receiving surface is inserted into the housing or is removed from the housing.

In one embodiment, a distance between one side of the receiving surface of the cartridge receiving portion and a portion of the sidewall of the housing facing the one side is greater than a thickness of the second plate region of the microstructure cartridge.

In one embodiment, the microstructure applicator may further comprise a band fastened to the housing for attaching the microstructure applicator to a user's body.

### Technical Effect

According to the microstructure cartridge and the microstructure applicator according to the present disclosure, the microstructure cartridge and a microstructure applicator which may easily check whether the microneedle has been used, thereby preventing the situation in which the microneedle that has already been used is reused, without confusion about whether the microneedle has been used.

### BRIEF DESCRIPTION OF THE DRAWINGS

FIG. 1 is a perspective view illustrating an embodiment of a microstructure cartridge according to the present disclosure.

FIG. 2 is a plan view of FIG. 1.

FIG. 3 is a perspective view illustrating another embodiment of a microstructure cartridge according to the present disclosure.

FIG. 4 is a plan view of a base plate in which the microstructure shown in FIG. 3 is omitted.

FIG. 5 is a perspective view illustrating a state in which the microstructure shown in FIG. 3 is received in an opening defined by a side barrier.

FIG. 6 is a plan view illustrating another example of a guide line of a base plate of a microstructure cartridge according to the present disclosure.

FIG. 7 is a perspective view illustrating an appearance of a microstructure applicator according to an embodiment of the present disclosure.

FIG. 8 is a perspective view illustrating a state in which a cartridge receiving portion of the microstructure applicator illustrated in FIG. 6 is opened.

FIG. 9 is a perspective view illustrating a state in which the microstructure cartridge shown in FIG. 8 is inserted into the cartridge receiving portion.

FIG. 10 is a perspective view illustrating a state in which the cartridge receiving portion shown in FIG. 9 is closed and a second plate region of the microstructure cartridge is bent.

FIG. 11 is a cross-sectional view taken along a line A-A′ of FIG. 10.

FIG. 12 is a view illustrating a state in which the microstructure shown in FIG. 11 is pressed such that a microneedle is inserted into the skin.

### DETAILED DESCRIPTIONS

Hereinafter, a microstructure cartridge and a microstructure applicator according to an embodiment of the present disclosure will be described in detail with reference to the accompanying drawings. The present disclosure may have various changes and may have various forms. Thus, specific embodiments will be illustrated in the drawings and will be described in detail herein. However, it should be understood that this is not intended to limit the present disclosure to a specific disclosure form and the present disclosure includes all modifications, equivalents, and replacements included in the spirit and technical scope of the present disclosure. While describing drawings, similar reference numerals are used for similar components. In the accompanying drawings, a dimension of a structure is shown to be larger than an actual one to make the clarity of the present disclosure.

It will be understood that, although the terms first, second, etc. may be used to describe various elements, the elements should not be limited by the terms. The terms are used only for the purpose of distinguishing one component from another component. For example, a first element could be termed a second element, and, similarly, a second element could be termed a first element, without departing from the scope of the present disclosure.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, and “including” when used in this specification, specify the presence of the stated features, integers, operations, elements, and/or components, but do not preclude the presence or addition of one or more other features, integers, operations, elements, components, and/or portions thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

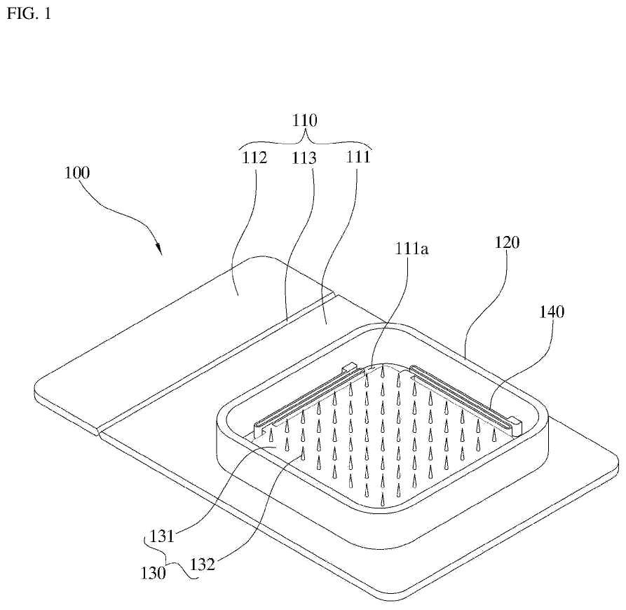

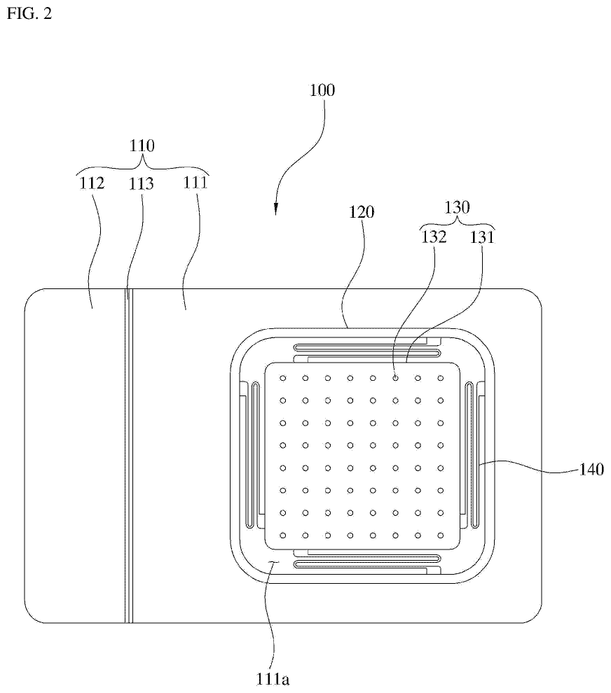

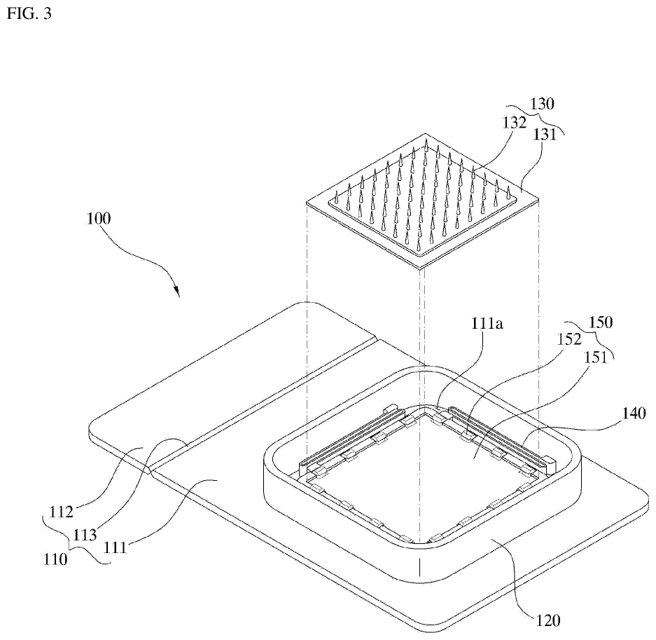

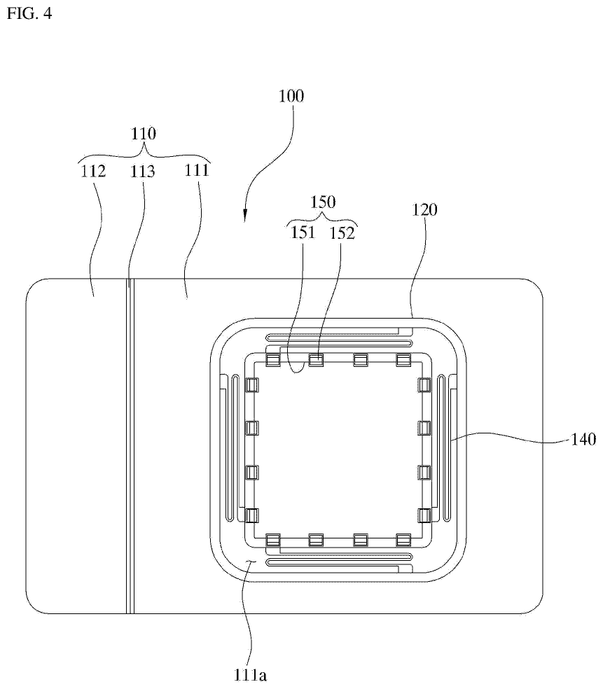

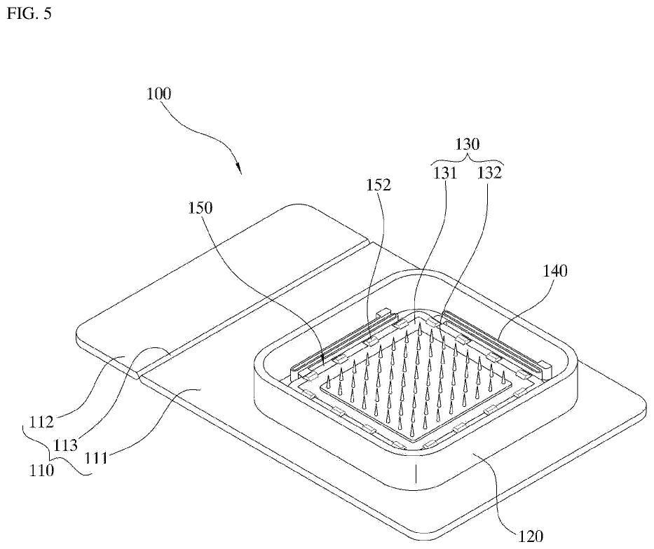

FIG. 1 is a perspective view illustrating one embodiment of a microstructure cartridge according to the present disclosure, FIG. 2 is a plan view of FIG. 1, FIG. 3 is a perspective view illustrating another embodiment of a microstructure cartridge according to the present disclosure, FIG. 4 is a plan view of a base plate in which the microstructure shown in FIG. 3 is omitted, and FIG. 5 is a perspective view illustrating a state in which the microstructure shown in FIG. 3 is received in an opening defined by a side barrier.

Referring to FIGS. 1 to 5, a microstructure cartridge 100 according to an embodiment of the present disclosure may include a base plate 110, a side barrier 120, and a microneedle 132.

The base plate 110 may include a first plate region 111, a second plate region 112, and a guide line 113.

The first plate region 111 is inserted into a microstructure applicator 200, which will be described later.

The second plate region 112 is a region that is not inserted into the microstructure applicator 200, which will be described later. The second plate region 112 is adjacent to the first plate region 111.

The guide line 113 acts as a boundary between the first plate region 111 and the second plate region 112, and guides bending or cutting of the second plate region 112.

For example, the base plate 110 may have a rectangular plate shape, the first plate region 111 may be a partial region of the rectangular plate shape, and the second plate region 112 may be the other partial region of the rectangular plate shape. In this case, the second plate region 112 may have a smaller size than that of the first plate region 111.

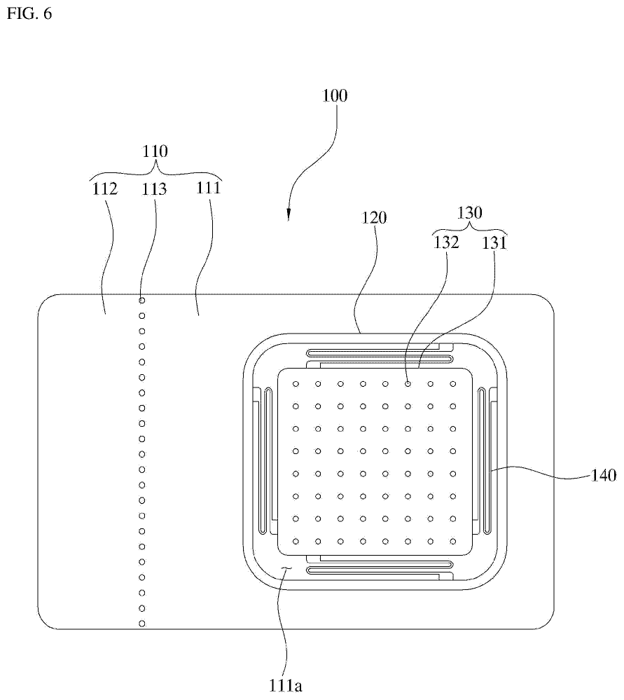

For example, as shown in FIGS. 1 to 5, the guide line 113 may be present in a form in which an engraved groove extends linearly in an upper surface and a lower surface of the base plate 110. In another example, as shown in FIG. 6, the guide line 113 may be present in a form in which a plurality of holes are arranged so as to be spaced from each other by a regular spacing.

The microneedle 132 is disposed on the first plate region 111, and a plurality of microneedles 132 are arranged on the first plate region 111. In this case, the microneedle 132 may be directly disposed on an upper surface of the first plate region 111 or may be provided as a separate microstructure.

For example, the microneedle 132 may be provided as a separate microstructure. In this case, the microstructure 130 may have a substrate 131, and a plurality of microneedles 132 may be arranged on an upper surface of the substrate 131.

In addition, when the microstructure 130 is provided, the first plate region 111 may include an opening 111a having a predetermined size and the side barrier 120 surrounding the opening 111a.

The microstructure 130 may be received in the opening 111a and may be surrounded with the side barrier 120.

The microstructure 130 may be supported on an inner surface of the side barrier 120 such that the microneedles 132 face a top end of the side barrier 120. In this case, the microstructure 130 may be elastically supported on the inner surface of the side barrier 120 to allow the microneedles 132 to be pressed toward the top end of the side barrier 120.

In an embodiment, as illustrated in FIGS. 1 and 2, the microstructure 130 may have a structure in which the substrate 131 is supported on the inner surface of the side barrier 120 via an elastic hinge 140. The elastic hinge 140 may have a structure in which an U-shape extends in a zigzag manner, and one end of the elastic hinge 140 extending in the zigzag manner may be connected to an end of the substrate 131 and the other end thereof may be connected to the inner surface of the side barrier 120. In this case, the elastic hinge 140 may be formed integrally with the side barrier 120 and the substrate 131.

In another embodiment, as shown in FIGS. 3 to 5, a microstructure receiving member 150 having a groove 151 into which the substrate 131 may be seated, and removal prevention protrusions 152 arranged along an edge of the groove 151 and contacting an upper surface of the substrate 131 seated in the groove 151 may be disposed so as to be surrounded with the side barrier 120. The elastic hinge 140 may be connected to each of four sides of the microstructure receiving member 150. The substrate 131 of the microstructure 130 may be seated in the groove 151.

The side barrier 120 may protrude upwardly from one surface of the first plate region 111. In this case, a vertical level of a top end of the side barrier 120 may be higher than a vertical level of a top end of the microneedles 132 of the microstructure 130 surrounded with the side barrier 120.

In the microstructure cartridge 100 according to an embodiment of the present disclosure, when the microstructure 130 is used so that the microneedles 132 penetrate into the skin, the second plate region 112 may be bent or cut at a boundary of the guide line 113 to indicate that the microstructure 130 is used.

In one example, the microstructure cartridge 100 according to an embodiment of the present disclosure may be used while being received in the microstructure applicator 200.

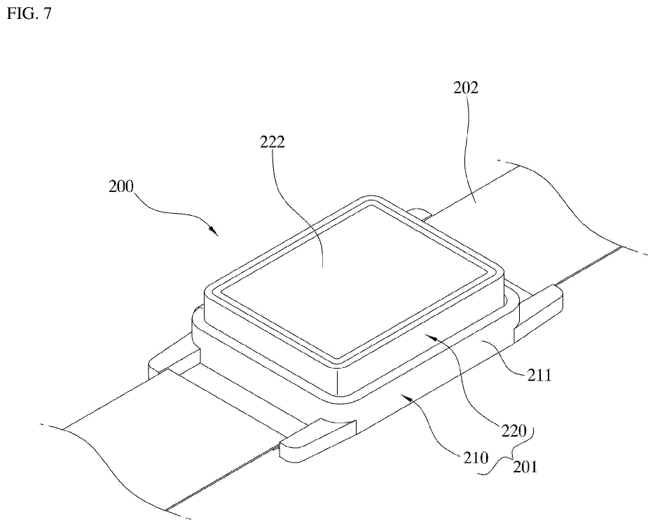

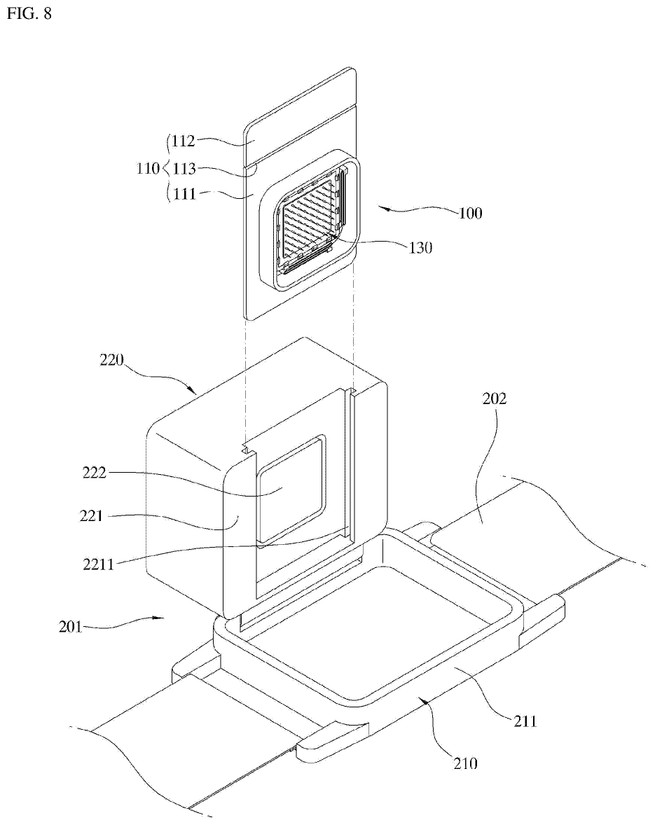

FIG. 7 is a perspective view illustrating an appearance of a microstructure applicator according to an embodiment of the present disclosure, and FIG. 8 is a perspective view illustrating a state in which a cartridge receiving portion of the microstructure applicator illustrated in FIG. 6 is opened.

Referring to FIGS. 7 and 8, the microstructure applicator 200 may include a housing 210 and a cartridge receiving portion 220.

The housing 210 has a sidewall 211 having a predetermined height, and upper and lower ends of the housing are opened. For example, the housing 210 may have a rectangular shape in a plan view.

The cartridge receiving portion 220 has a cartridge receiving surface 221 for accommodating the microstructure cartridge 100 therein, and the cartridge receiving surface 221 is inserted into the housing 210. The cartridge receiving portion 220 may be sized so as to be inserted into the housing 210.

In this regard, when the cartridge receiving surface 221 is inserted into the housing 210, a distance between one side of the cartridge receiving surface 221 of the cartridge receiving portion 220 and the sidewall 211 of the housing 210 facing the one side is greater than the thickness of the second plate region 112 of the microstructure cartridge 100. This configuration provides a space into which the second plate region 112 may be bent so as to be positioned, between the sidewall 211 and the one side of the receiving surface 221 facing each other.

In one example, a slot 2211 may be defined in the cartridge receiving surface 221, and a pressing button 222 may be provided in the cartridge receiving portion 220.

The slot 2211 has a width and a length sized such that the first plate region 111 of the microstructure cartridge 100 may be inserted into the slot 2211.

The pressing button 222 is disposed above the slot 2211 and pressed toward the slot 2211. Although not shown in detail, the pressing button 222 may be configured to be returned after being pressed toward the slot 2211.

In one example, the cartridge receiving portion 220 is configured such that one side of the cartridge receiving surface 221 is hingedly connected to the sidewall 211 of the housing such that the cartridge receiving surface 221 is inserted into the housing 210 or is removed out of the housing 210.

As described above, the distance between the one side of the receiving surface 221 of the cartridge receiving portion 220 and the sidewall 211 of the housing 210 facing each other may be greater than the thickness of the second plate region 112 of the microstructure cartridge 100. In this regard, the one side of the receiving surface 221 may be opposite to the side of the receiving surface 221 at which the cartridge receiving portion 220 is hingedly connected to the sidewall 211 of the housing.

Further, a band 202 may be coupled to the housing 210 and may be used to attach the microstructure applicator 200 to a user's body.

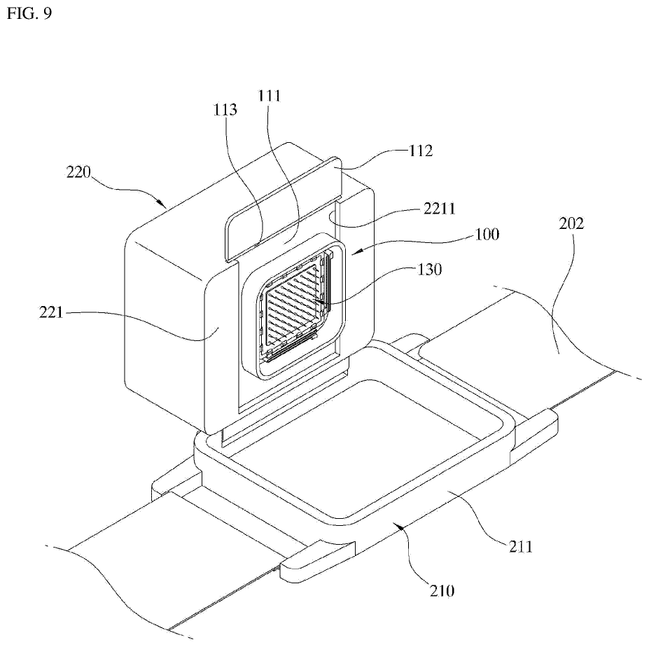

FIG. 9 is a perspective view illustrating a state in which the microstructure cartridge shown in FIG. 8 is inserted into the cartridge receiving portion.

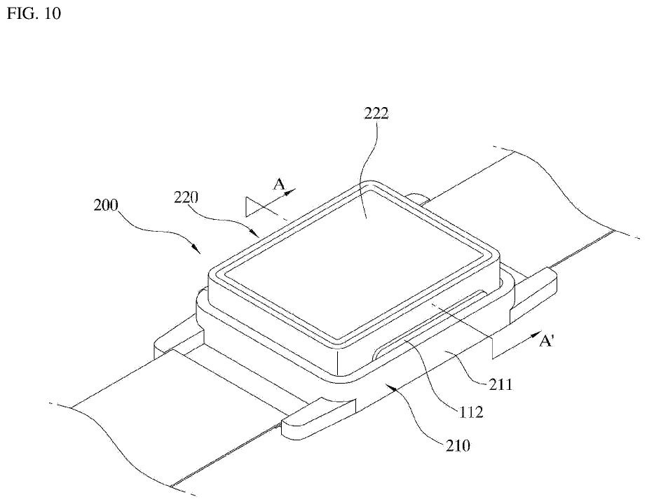

When the microstructure cartridge 100 is inserted into the cartridge receiving portion 220, the first plate region 111 is inserted into the slot 2211, and the second plate region 112 is not entirely received in the cartridge receiving portion 220 but is partially exposed to the outside, as shown in FIG. 10. Accordingly, when the cartridge receiving portion is received in the housing 210, the second plate region may be engaged with the sidewall so as to be bent or cut.

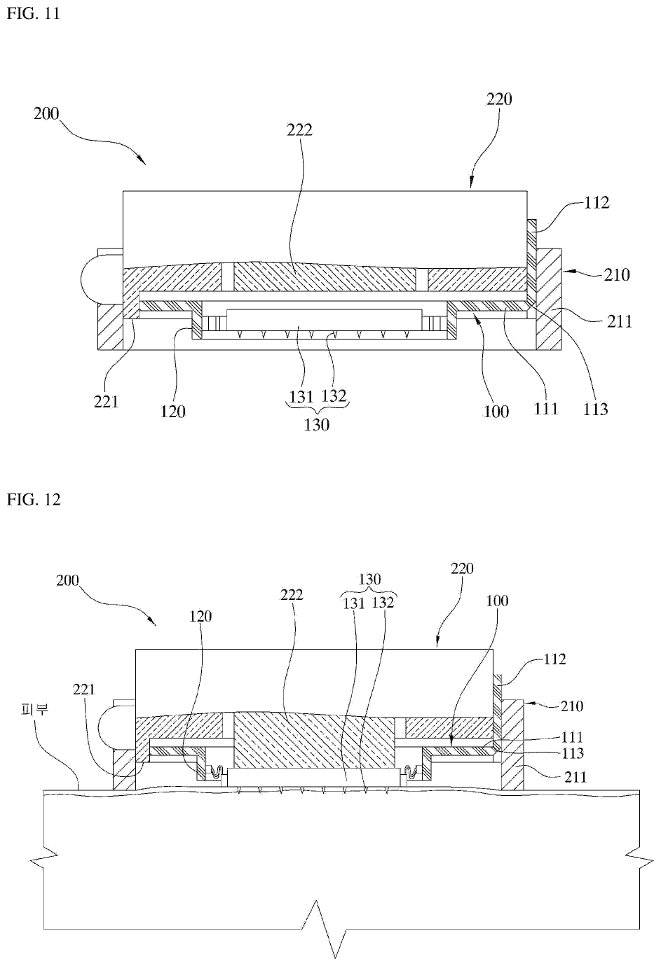

Hereinafter, a process of delivering a drug to the skin of a user by using the microstructure applicator according to an embodiment of the present disclosure will be described with reference to FIGS. 8 to 11. FIG. 10 is a perspective view illustrating a state in which the cartridge receiving portion shown in FIG. 9 is closed and the second plate region of the microstructure cartridge is bent, and FIG. 11 is a cross-sectional view taken along a line A-A′ of FIG. 10. FIG. 12 is a view illustrating a state in which the microstructure shown in FIG. 11 is pressed such that the microneedle is inserted into the skin.

First, in a state in which the microstructure applicator 200 is worn on the wrist of a user and the cartridge receiving portion 220 is spaced apart from the housing 210 and is open, as shown in FIG. 8, the first plate region 111 of the base plate 110 of the microstructure cartridge 100 is inserted into the slot 2211 of the cartridge receiving portion 220 as shown in FIG. 9. In this case, the second plate region 112 adjacent to the first plate region 111 is not inserted into the slot 2211 but is exposed out of the cartridge receiving portion 220, and the guide line 113 is positioned at or close to an edge of one side of the cartridge receiving portion 220.

Subsequently, the cartridge receiving portion 220 is pushed toward the housing 210 and inserted into the housing 210. At this time, when the cartridge receiving portion 220 is inserted into the housing 210, the second plate region 112 is pressed into a gap between the side surface of the cartridge receiving portion 220 and the inner surface of the sidewall 211, and thus is bent from the first plate region 111 at and along the guide line 113.

As shown in FIGS. 10 and 11, the second plate region 112 is bent by 90 degrees so as to be inserted into the gap between the side surface of the cartridge receiving portion 220 and the inner surface of the sidewall 211 as shown in FIGS. 10 and 11.

In addition, the microneedle 132 of the microstructure 130 faces the user's skin and is exposed out of the open lower end of the housing 210.

Finally, when the pressing button 222 is pressed, as shown in FIG. 12, the substrate 131 of the microstructure 130 is pressed downward so that the microneedle 132 is inserted into the skin of the user. At this time, the elastic hinge 140 is deformed to be stretched such that the substrate 131 of the microstructure 130 may be lowered toward the skin of the user.

Thus, the microneedle 132 is inserted into the user's skin and the drug therefrom is delivered to the skin. Then, the microstructure cartridge 100 is removed from the microstructure applicator 200. At this time, when the user pulls the cartridge receiving portion 220 upwardly, the cartridge receiving portion 220 is opened such that the microstructure 130 is spaced apart from the skin of the user.

In the microstructure cartridge 100 removed from the microstructure applicator 200, the second plate region 112 of the base plate 110 is in a bent state from the first plate region 111. Thus, this state indicates that the microstructure cartridge 100 has been used.

When the microstructure cartridge and the microstructure applicator according to an embodiment of the present disclosure are used, the user thereof may easily check whether the microneedle 132 has been used, thereby preventing the situation in which the microneedle 132 as already used is reused, without confusion about whether the microneedle 132 has been used.

In addition, the microstructure applicator 200 is worn on the wrist of the user using the band 202 and thus comes into close contact with the skin, so that the skill may fill the housing 210 of the microstructure applicator 200 such that the microneedle 132 may be more easily inserted into the skin of the user.

The description of the disclosed embodiments is provided to enable a person skilled in the art to make or use the present disclosure. Various modifications to these embodiments will be apparent to one of ordinary skill in the art, and the general principles defined herein may be applied to other embodiments without departing from the scope of the disclosure. Thus, the present disclosure is not intended to be limited to the embodiments presented herein but should be construed in the widest scope consistent with the principles and novel features presented herein.

1. A microstructure cartridge comprising:
a base plate including:
a first plate region to be inserted into a microstructure applicator;
a second plate region adjacent to the first plate region and constructed not to be inserted into the microstructure applicator; and
a bending or cutting guide line as a boundary between the first plate region and the second plate region; and
a microneedle disposed on the first plate region of the base plate,
wherein the second plate region is constructed to be bent or cut along the guide line.

2. The microstructure cartridge of claim 1, wherein the guide line is formed in a groove extending linearly along one direction.

3. The microstructure cartridge of claim 1, wherein the guide line includes a linear arrangement of holes arranged so as to be spaced from each other by a predetermined spacing along one direction.

4. A microstructure applicator comprising:
a housing having a sidewall having a predetermined height, wherein upper and lower ends of the housing are opened; and
a cartridge receiving portion having a cartridge receiving surface for receiving the microstructure cartridge of claims 1 therein, wherein the cartridge receiving surface is constructed to be inserted into the housing,
wherein the second plate region is exposed to an outside while not being received in the cartridge receiving portion,
wherein when the cartridge receiving portion is received in the housing, the second plate region is engaged with the sidewall so as to be bent or cut.

5. The microstructure applicator of claim 4, wherein one side of the cartridge receiving surface is hingedly connected to the sidewall such that the cartridge receiving surface is inserted into the housing or is removed from the housing.

6. The microstructure applicator of claim 4, wherein a distance between one side of the receiving surface of the cartridge receiving portion and a portion of the sidewall of the housing facing the one side is greater than a thickness of the second plate region of the microstructure cartridge.

7. The microstructure applicator of claim 4, comprising a band fastened to the housing for attaching the microstructure applicator to a user's body.
